(19) United States
(12) Patent Application Publication
(10) Pub. No.: US 2026/0306026 A1
(43) Pub. Date: Oct. 8, 2026

(54) REAL-TIME BLADDER SENSING SYSTEM THROUGH BIO-IMPLANTABLE SENSOR ELEMENT

(71) Applicants: KOREA ADVANCED INSTITUTE OF SCIENCE AND TECHNOLOGY, Daejeon (KR); POSTECH RESEARCH AND BUSINESS DEVELOPMENT FOUNDATION, Gyeongsangbuk-do (KR)

(72) Inventors: Steve Park, Daejeon (KR); Sung-Min Park, Gyeongsangbuk-do (KR); Byungkook Oh, Daejeon (KR); Young-Soo Lim, Gyeongsangbuk-do (KR)

(21) Appl. No.: 19/108,238

(22) PCT Filed: Aug. 11, 2023
(86) PCT No.: PCT/KR2023/011978

§ 371 Date: Mar. 3, 2025

(30) Foreign Application Priority Data
Nov. 15, 2022 (KR) 10-2022-0152858
Jul. 24, 2023 (KR) 10-2023-0096402

(51) Int. Cl. A61B 5/00; A61B 5/20; A61B 5/391; A61B 5/397

(52) U.S. Cl. CPC A61B 5/688; A61B 5/205; A61B 5/391; A61B 5/397; A61B 5/6874; A61B 2562/0261

(57) ABSTRACT

The present invention discloses a real-time bladder sensing system using a bio-implantable sensor device including a sensor unit attached to a bladder, and an analysis unit configured to analyze a signal received from the sensor unit, wherein the analysis unit calculates a root-mean-square deviation of the EMG sensing signal to determine a urination time point and tracks a change in the number of urination time point thereby determining whether bladder is overactive or not.

### TECHNICAL FIELD

The present invention relates to a real-time bladder sensing system through a bio-implantable sensor device, and more particularly, to a real-time bladder sensing system through a bio-implantable sensor device which can monitor a physiological phenomenon of a bladder in real time using a biocompatible element.

### BACKGROUND ART

The human urinary bladder is a musculomembranous pouch that functions as a urinary reservoir, located in the anterior portion of the pelvic cavity, which receives urine through the ureter and excretes through the urethra. In humans, the bladder is in the pelvic bone posterior pelvis (the pubic symphysis) and is connected to the superior posterior segment of the bladder by a vasculature called the urethra, which escapes the body. Urinary bladder is subject to various diseases and injuries that worsen urinary bladder in patients. For example, bladder deterioration may be due to infectious diseases, neoplasms, and developmental malformations.

Therefore, there is a need for the development of real-time monitoring technology for the treatment and prevention of such bladder diseases.

[Prior Art] (Non-Patent Document 1) Expandable and Implantable Bioelectronic Complex for Analyzing and Regulating Realtime Activity of the Urinary Bladder Paper DOI: https://advances.sciencemag.org/content/6/46/eabc9675 (2020 Nov. 11).

### DISCLOSURE OF THE INVENTION

### Technical Problem

Accordingly, an object to be achieved by the present disclosure is to provide a real-time bladder sensing system and method.

### Technical Solution

In order to solve the above problems, the present invention provides a real-time bladder sensing system using a bio-implantable sensor device, the real-time bladder sensing system comprising: a sensor unit attached to the bladder; and an analysis unit for analyzing a signal received from the sensor unit.

In an embodiment of the present invention, the sensor unit may simultaneously sense EMG and strain.

In one embodiment of the present invention, the sensor unit is formed on a hydrogel substrate containing polyacrylamide, and is attached to the bladder through the hydrogel substrate.

In an embodiment of the present disclosure, the analysis unit filters a peak signal from the strain sensing signal and then predicts a urination point therefrom.

The analysis unit may calculate a root-mean-square deviation of the EMG sensing signal, determine a urination time point, track a change in the number of urination, and predict whether the bladder has an overactive bladder.

In an embodiment of the present invention, the analysis unit determines the EMG RMS signal point having a value of 2×(average EMG RMS) or more as the urination time point.

In one embodiment of the present invention, the analysis unit receives the EMG, and strain signals and integrates them to predict the physiological phenomenon of the bladder.

In one embodiment of the present disclosure, the physiological phenomenon of the bladder is the time point of urination and whether the bladder is overactive or not.

In one embodiment of the invention, the EMG sensor comprises three electrodes formed on a radial island substrate, the radial island substrate being bonded onto the hydrogel substrate.

In one embodiment of the present invention, a swelling prevention coating layer is formed on the sensor unit.

In an exemplary embodiment of the present disclosure, the swelling prevention coating layer includes an acryl gate-based material, and the real-time bladder sensing system has a lower modulus than that of the human body bladder.

The invention also provides a sensor for the bladder sensing system described above, wherein the sensor is adhered to the bladder via a hydrogel-based adhesive.

### Advantageous Effects

The present invention provides a sensor system which is biocompatible and has adhesive properties, wherein the sensor system can accurately monitor the physiology of the bladder using biocompatible materials. The sensor system according to the present invention is made of soft materials having superior biocompatibility so as to use materials which can minimize side effects when inserted into the body, and can operate reliably even when mechanical stimulation is applied while minimizing reaction speed and hysteresis.

### BRIEF DESCRIPTION OF THE DRAWINGS

FIG. 1 is a schematic diagram illustrating a manufacturing method according to an embodiment of the present invention.

FIG. 2 is a photograph after a sensor manufactured according to the present invention is attached to the bladder.

FIG. 3 is a block diagram of a sensing system according to the present invention.

FIG. 4 is a diagram illustrating a sensing system and a sensing method manufactured according to an embodiment of the present invention.

FIG. 5 is a graph showing relative volume changes in PBS with and without Anti-swelling coatings.

FIG. 6 is a strain-stress curve of PAAm with or without an anti-swelling coating.

FIG. 7 is an adhesive strength graph in the case of using a conventional silicone rubber (Ecoflex-0020) as an adhesive and in the case of using a hydrogel-based adhesive on the same substrate (PAAm).

FIG. 8 is a result of testing in vitro cytotoxicity of strain sensors and EMG sensor materials formed on a hydrogel substrate using HEK293 cells.

FIG. 9 is an In-vivo protocol image of contraction and expansion of the bladder using a rat model.

FIG. 10 is a sensing result of a strain sensor and an EMG sensor attached to the bladder by inducing a repeated urination process through injection and discharge of liquid by connecting a rubber tube to the rat bladder.

FIG. 11 illustrates a result of improving Signal to Noise ratio (SNR) as compared with the existing EMG sensor by coating platinum black (PtB) on the EMG sensor of the sensor system according to the present invention to increase the entire cross-sectional area of the sensor.

FIG. 12 illustrates a result showing higher EMG activity in a bladder swelling region when comparing a normal bladder region and a bladder swelling region.

FIG. 13 shows the results of tracking the amount and time of urination before (normal state) and after (overactive bladder-induced state) the infusion of overactive bladder-induced drug.

FIG. 14 is a result of comparing and analyzing the results of FIG. 13 with one-time urination amount and urination time in the normal section and the overactive bladder induction section.

FIG. 15 shows a result of tracking physiological indicators (urination amount, bladder internal pressure) in a civil bladder induction section.

FIG. 16 shows measurement results of a tensile sensor and an EMG sensor in an overactive bladder induction region.

FIG. 17 is a result of extracting data near the urination time point and averaging it based on the urination time point.

FIG. 18 is a result of quantitatively analyzing the result of FIG. 17.

FIG. 19 is an image of a surgical protocol in which a hydrogel-based implantable sensor developed by the present researchers is implanted using a minimally invasive laparoscopic robot implantable surgical instrument.

FIG. 20 is an image of an actual minimally invasive laparoscopic robot implant surgery using a pig.

FIG. 21 is an image showing a surgical procedure in which a hydrogel-based implantable sensor developed by the present researchers using a laparoscopic robot implant surgery is implanted and attached to the bladder of a pig.

FIG. 22 is an image showing a repeated urination process of a pig, and the results of strain sensors and EMG sensors sensed in the urination process reflect the actual urination process of a pig.

### BEST MODE FOR CARRYING OUT THE INVENTION

In order to solve the above-described problems, the present invention provides a method and a system capable of analyzing the physiological phenomenon of an intractable bladder model by integrating a sensor capable of evaluating mechanical properties and electrical properties into a hydrogel having biocompatibility and adhesion properties.

A material used as a bio-implantable sensor for analyzing and controlling a physiological phenomenon of the bladder, which has been reported in the related art, Non-Patent Document 1, is a silicone rubber. Such material has a hydrophobic surface, and has a disadvantage in that Young's modulus (several MPa to several GPa) is significantly different from that of a bio-organ (several Pa to several KPa), and there is no functional group that can be attached to the surface of the bio-organ, and thus the adhesive properties are not good. Therefore, due to the disadvantage, an additional fixing system is required to adhere to a bio-insertion sensor capable of analyzing a physiological phenomenon to the organ surface.

The problem is that, when such a fixing system is used, reliability of a sensor signal is deteriorated because an air layer is formed by being easily peeled off from a surface of a living body, and there is a disadvantage in that a new fixing stand should be designed for use in various organs.

Therefore, in an embodiment of the present invention, in order to develop a biocompatible sensor capable of monitoring physiological phenomena of the bladder having an incurable disease in real time, a polyacrylamide-based hydrogel material having a mechanical property (Young's modulus: \tilde{ }300 kPa) similar to that of the tissue of the bladder was used as a substrate material. The polyacrylamide used in an embodiment of the present invention has a water content of 80 to 90% or more and has Amine-group capable of chemically bonding with various biological tissues, and thus has excellent adhesion characteristics with tissues compared to a silicone rubber-based material which is widely used.

In addition, two types of sensors were simultaneously used as sensors on the hydrogel material substrate. That is, the sensor according to the present invention includes a strain sensor for measuring contraction and relaxation, and an EMG sensor for measuring bioelectricity of a nerve signal causing detrusor muscle outflow. Accordingly, one sensor can stably adhere to the bladder tissue to measure both strain in the bladder and an EMG signal and thus can monitor neural activity for more comprehensive and accurate urination detection.

Hereinafter, the present invention will be described in more detail with reference to Examples.

### Embodiments

### Manufacture of Polyacrylamide (PAAm) Substrates

Materials for the PAAm substrate were prepared by synthesizing 4.8 g of acrylamide (PAAm, SigmaAldrich), 0.0038 g of N,N′-methylenebisacrylamide as a curing agent (MBA, Sigma-Aldrich), and 0.06 g of 2-Hydroxy-4′-(2-hydroxyethoxy)-2-methylpropiophenone as a photoinitiator (Irgacure-2959 powder, Sigma-Aldrich) in UCP-10 of water diluted with ultrasonic waves (JEIO TECH, 40 Hz, 10 mL). The as-synthesized 100 μL PAAm solution was drop-cast onto a carrier substrate (diced Si wafer, 10 mm×15 mm), and then the coated PAAm solution was cured with ultraviolet (UV) light for 1 minute.

### EGaIn (Eutectic Gallium/Indium Liquid Metal) Synthesis

EGaIn ink was synthesized by mixing 0.3 mL of bulk eutectic gallium indium alloy (EGaIn (LM), Rich-Metals) and diluted acetic acid (3 vol % in 3 mL diluted water) in a vial, followed by tip ultrasonication (VC 505, Sonics & Materials, 3 mm microtip) at 500 W. and 20 kHz. for 10 minutes.

### PAAm Printing Which Is a Strain Sensor on an EGaIn Substrate

A nozzle printer (BIO X6, CELLINK) was used for meniscus guide coating of the PAAm substrate to print the EGaIn on the PAAm substrate. The prepared EGaIn ink was placed in a syringe used for direct nozzle printing and the printing bed was heated to 60° C. The tip diameter and printing speed of the nozzle were 200 μm and 6.4 mm/s, respectively, and after coating the PAAm substrate with EGaIn, an anisotropic conductive film was attached to an electrical pad made of EGaIn, and then a PI conductive wire was connected on the electrical pad. As an encapsulation layer, the PAAm thin film was coated through drop casting of 20 μL PAAm solution to the strain sensor and the electrical pad area. In addition, an anti-swelling layer was coated on the PAAm substrate. This will be described in more detail below.

### Swelling Prevention Layer Coating

Coating the swelling prevention layer on the PAAm substrate includes a process of mixing an acrylate-based material and an ultraviolet (UV) photo-reactive primer in an organic solution. In an exemplary embodiment of the present invention, the material of the polymerization unit derived from dodecyl acrylate is used, but any one polymerization unit selected from the group consisting of the polymerization unit derived from stearyl acrylate and a combination thereof may be included. That is, in order to diffuse the functional primer of HMPP (2-hydroxy-2-methylpropiophenone) into the PAAm substrate, the PAAm substrate was immersed in the prepared primer solution for 10 minutes and then rinsed with ethanol and diluted water. Thereafter, UV light was applied to the PAAm substrate to form a thin swelling prevention layer on the PAAm substrate. The effects of the swelling prevention layer according to the present disclosure will be described in more detail below.

### Manufacture of Electromyography (EMG) Sensors

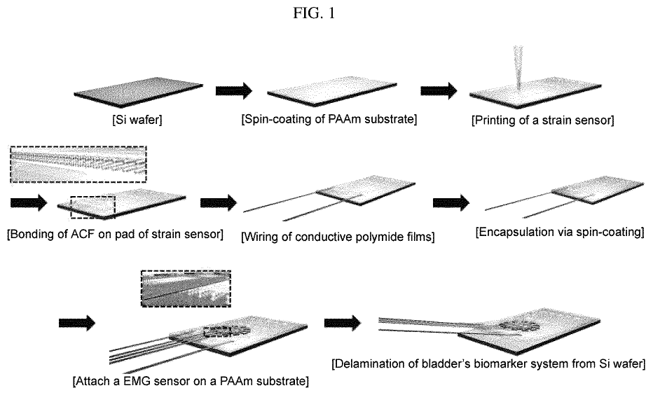

In order to manufacture an electromyogram sensor capable of sensing a mechanical force, an islet shaped like a spectator having a maximum output of 50 W was patterned on a polyimide film (PI, thickness: 50 μm) using a CO2 laser cutter (CE6000-60, GRAPHTEC) (see FIG. 1). The maximum scan speed was about 500 mm/s under ambient conditions. Ti/Au (10 nm/200 nm) was deposited on a PI island substrate in the shape of an orchard using an E-beam evaporator (SNTEK) with a custom metal magnetic mask. The electrodes deposited on the equatorial island-shaped electrodes were composed of reference, recording and ground electrodes and also included electrical pads for external connections. Thereafter, platinum black was coated on the counter, recording, and ground electrodes. An anisotropic conductive film was attached to the electrical pads, and then PI conductive wires were connected to each of the three electrical pads. That is, the radial type electrode sensor for a viewing car according to the present invention has excellent contractility and tensile properties, which will be described in more detail below.

### Integration of Electromyography (EMG Sensor) Into a Hydrogel Substrate

A hydroxyl device was formed on the bottom surface (a surface without an electrode) of the island of the EMG sensor in the shape of a large viewing car described above by oxygen plasma treatment at 100 W for 1 minute. Thereafter, the reaction mixture was subjected to chemical vapor deposition (CVD) of a 10 vol % (2-Hydroxy-4′-(2-hydroxyethoxy)-2-methylpropiophenone) (Irgacure-2959, Sigma-Aldrich) solution in ethanol at 60° C. in a vacuum oven (OV3-30, JEIO TECH) for 1 hour, and then, after the CVD, a PAAm solution was coated on the surface of the island. After applying UV light for 5 minutes, an island-shaped EMG sensor was attached to the PAAm substrate.

### Bioadhesive Synthesis

For smooth interaction with the bladder, a hydrogel-based adhesive was prepared by mixing acrylamide 4.8 g, CaCl (Sigma-Aldrich) 0.712 g, ammonium persulfate (AMPS, Sigma-Aldrich) 0.0248 g, and k-carrageenan (Sigma-Aldrich) 0.05 g.

FIG. 1 is a schematic diagram illustrating a manufacturing method according to an embodiment of the present invention.

Referring to FIG. 1, it can be confirmed that the linear strain sensor having a plurality of curved U-shaped curved portions according to the above-described steps and the EMG sensor composed of three electrodes formed on the viewer's vehicle-shaped polyimide substrate having a structure in which islands are radially radiated from the central circular portion are simultaneously implemented on the hydrogel substrate (PAAm). In the present invention, an island EMG sensor in the shape of a viewing car was used to secure mechanical stability of the sensor against repeated contraction and expansion in the urination process of the bladder.

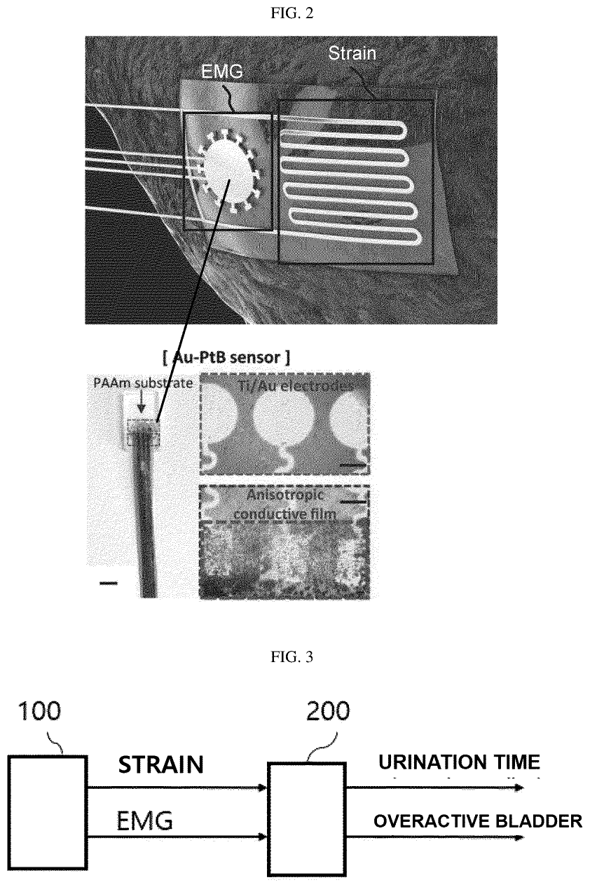

FIG. 2 is a photograph after a sensor manufactured according to the present invention is attached to the bladder.

Referring to FIG. 2, it can be seen that an island EMG sensor in the shape of a viewing car and a strain sensor in the shape of a line having a plurality of bent portions are simultaneously implemented on one hydrogel substrate. An island EMG sensor in the shape of a viewing car was used to secure mechanical stability of the sensor against repeated contraction and expansion in the urination process of the bladder. In addition, the lower image of FIG. 2 is an image of an EMG sensor, and is an image in which it can be confirmed that three electrodes are formed on an island-shaped polyimide substrate in which a plurality of radial structures protrude.

FIG. 3 is a block diagram of a sensing system according to the present invention.

Referring to FIG. 3, the real-time bladder sensing system according to an embodiment of the present invention includes the sensor unit 100 of FIG. 2 attached to the bladder; and the analysis unit 200 for analyzing a signal received from the sensor.

Describing the operation of the analyzing unit 200 of FIG. 3 in more detail, after filtering the peak signal from the strain sensing signal, the urination point is predicted from the peak signal. Also, the analysis unit 200 may calculate a root-mean-square deviation of the EMG sensing signal and then predict whether the EMG sensing signal is an overactive bladder. Accordingly, the present invention can utilize two types of signals obtained from a sensor that can be attached to the bladder muscle itself, not the vicinity of the bladder, for measurement of urination point, analysis of overactive bladder, etc.

### Experimental Example

### Hydrogel-Based In Vivo Implantable Sensor Intractable Bladder Model Analysis Process Analysis

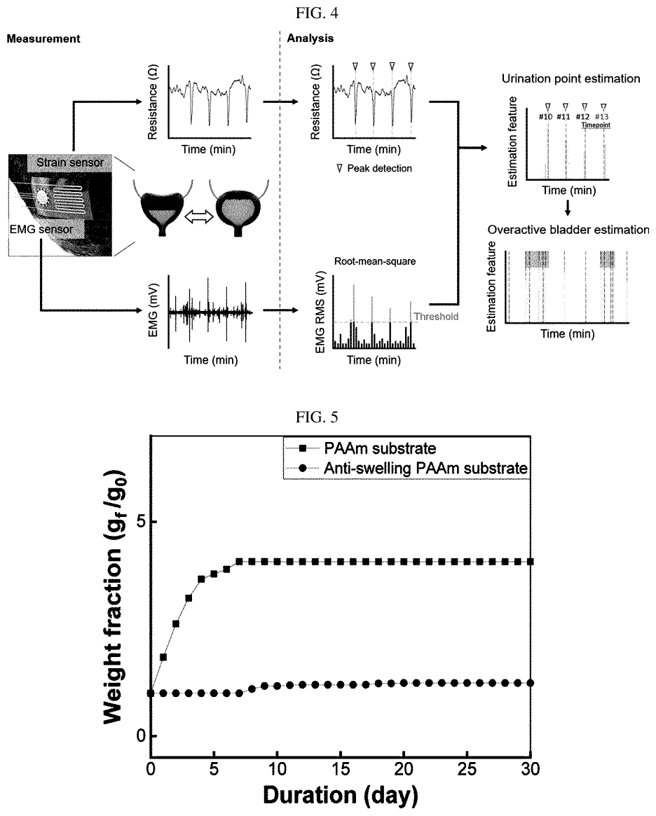

FIG. 4 is a diagram illustrating a sensing system and a sensing method manufactured according to an embodiment of the present invention.

Referring to FIG. 4, the sensor manufactured according to an embodiment of the present disclosure may be attached to the bladder on the hydrogel substrate to simultaneously sense the EMG and the strain and may integrate the same to analyze the phenomenon of the bladder (urination point estimation and overreactive bladder estimation) in real time.

FIG. 5 is a graph showing relative volume changes in PBS with and without Anti-swelling coatings.

Referring to FIG. 5, it can be seen that when there is no swelling prevention coating layer, a weight change was nearly 4 times generated after 1 week. However, it can be seen that when there is the anti-swelling coating layer according to the present invention, there is a volume change of almost negligible level (\tilde{ }1.2 times) even after 30 days.

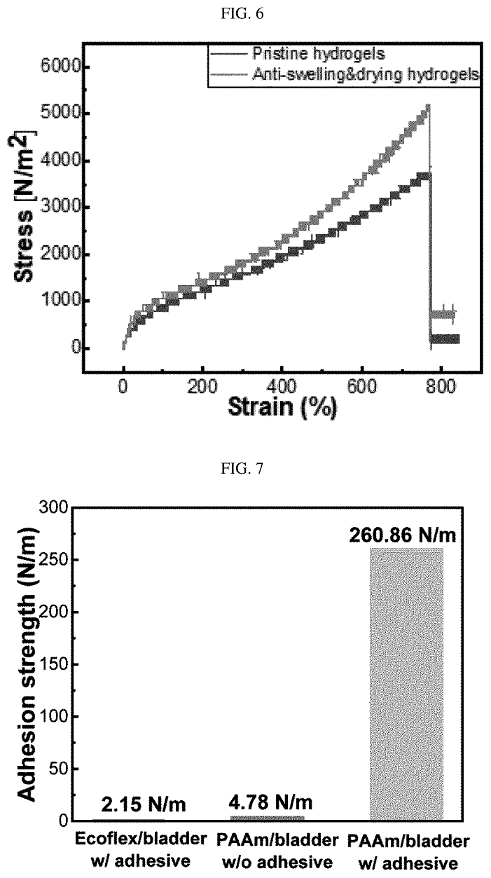

FIG. 6 is a strain-stress curve of PAAm with or without an anti-swelling coating.

Referring to FIG. 6, it can be seen that even when there is the anti-swelling coating, the Young's modulus of the substrate is still very low (0.8 to 1 kPa) than the modulus (\tilde{ }300 kPa) of the bladder. Therefore, this result means that the sensor according to the present invention has a very low physical influence on the natural movement of the bladder. That is, this result proves that the mechanical properties of the existing PAAm are not significantly changed even when the swelling prevention layer that prevents weight change in vivo according to the present invention is coated.

FIG. 7 is an adhesive strength graph in the case of using a conventional silicone rubber (Ecoflex-0020) as an adhesive and in the case of using a hydrogel-based adhesive on the same substrate (PAAm).

Referring to FIG. 7, the PAAm substrate according to the present disclosure showed higher adhesive strength (260.86 N/m) than the silicone rubber substrate (2.15 N/m) in the related art. That is, a silicone rubber-based rubber material, which is widely used, exhibits a characteristic in which bladder and adhesive strength are not good even when an adhesive is used. The silicon rubber-based rubber material has an adhesive force material containing a large amount of —OH groups on the surface thereof and a chemical functional group capable of being bonded to the surface of the bladder, and thus the adhesive force is lower than the adhesive force of PAAm without using the adhesive force. However, the adhesive strength of the PAAm using the adhesive material according to the present invention is hundreds of times higher than that of the bladder, and this is a result showing that stable adhesion can be maintained during contraction and expansion during a urination process of the bladder.

### Evaluation of In-Vivo Characteristics of a Sensor Integrated Into a Hydrogel Material

The present invention can implement a system capable of monitoring physiological phenomena of the bladder having an intractable model in real time by integrating a sensor capable of evaluating mechanical and electrical characteristics with hydrogel-based material. In addition, since an excellent adhesive with human tissue such as the bladder is used, it is possible to stably bond the pig by inserting the pig into the bladder without additional suturing surgery through minimally invasive laparoscopic robotic surgery.

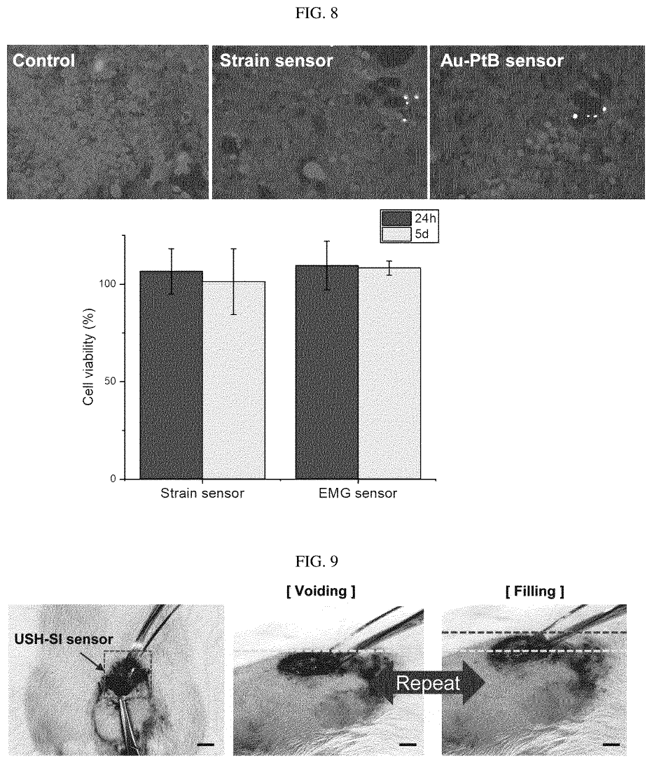

FIG. 8 is a result of testing in vitro cytotoxicity of strain sensors and EMG sensor materials formed on a hydrogel substrate using HEK293 cells.

Referring to FIG. 8, the sensor (USH-SI) according to the present invention exhibited in vitro cytotoxicity at a level comparable to that of a control group (pure HEK293 cells cultured with a 48-well platter in Dulbecco Modified Eagle Medium, DMEM).

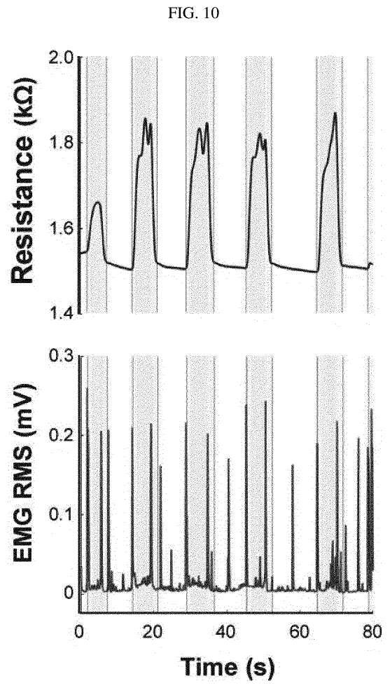

FIG. 9 is an In-vivo protocol image of contraction and expansion of the bladder using a rat model, and FIG. 10 is a sensing result of a strain sensor and an EMG sensor attached to the bladder by inducing a repeated urination process through injection and discharge of liquid by connecting a rubber tube to the rat bladder.

Referring to FIGS. 9 and 10, it can be seen that the injection of the vegetation water increased the resistance of the strain sensor, and the contraction decreased the resistance in the opposite direction.

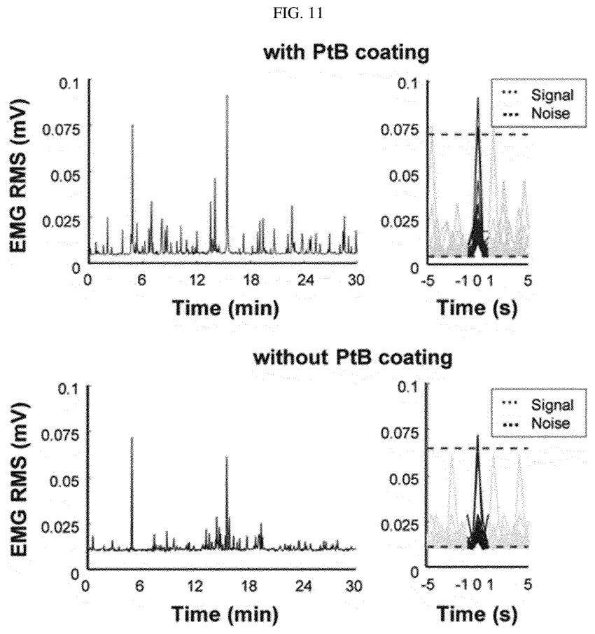

FIG. 11 illustrates a result of improving Signal to Noise ratio (SNR) as compared with the existing EMG sensor by coating platinum black (PtB) on the EMG sensor of the sensor system according to the present invention to increase the entire cross-sectional area of the sensor. In the present experimental example, the SNR was calculated by using a ratio of the average RMS signal and the RMS noise (SNR (dB)=10 log (PowerSignal/PowerNoise)=20 log (AmplitudeSignal (RMS)/AmplitudeNoise(RMS)).

Referring to FIG. 11, an EMG sensor coated with platinum black (PTB) according to an embodiment of the present disclosure showed an SNR of 22.84 dB superior to that of a case without a PTB coating. This result indicates that the PTB coating is useful for increasing the signal to noise ratio of the EMG sensor in vivo conditions.

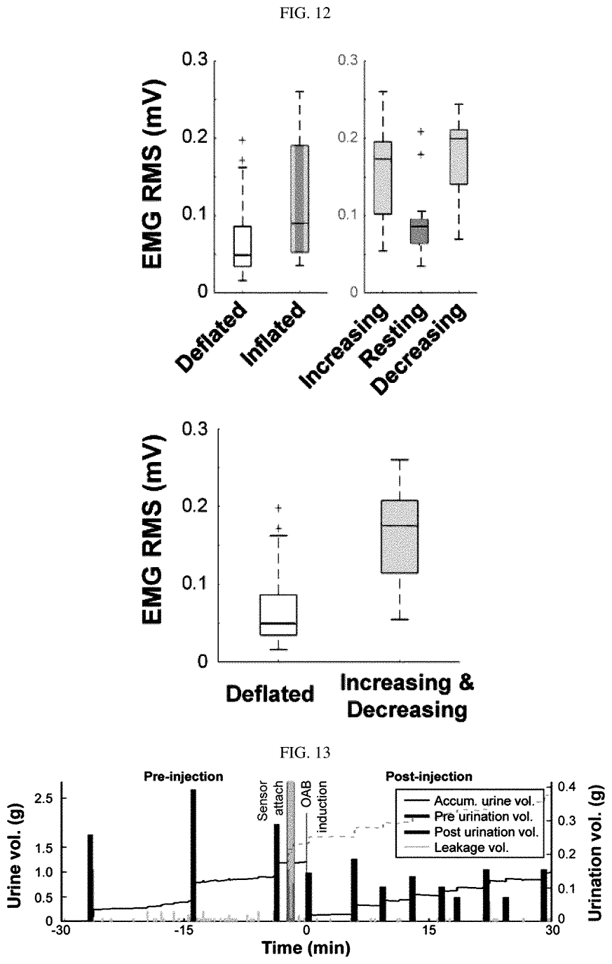

FIG. 12 illustrates a result showing higher EMG activity in a bladder swelling region when comparing a normal bladder region and a bladder swelling region.

Referring to FIG. 12, the box plot analysis result of EMG RMS varies depending on the bladder state, and the box plot according to the bladder capacity is displayed at the upper left, and the box plot according to the other transition state is displayed at the upper right.

FIG. 13 shows the results of tracking the amount and time of urination before (normal state) and after (overactive bladder-induced state) the infusion of overactive bladder-induced drug.

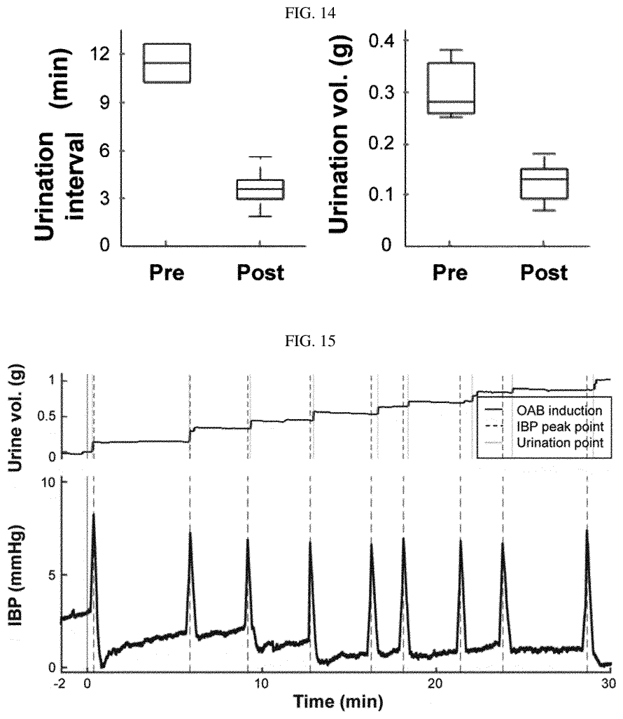

FIG. 14 is a result of comparing and analyzing the results of FIG. 13 with one-time urination amount and urination time in the normal section and the overactive bladder induction section.

In FIG. 15, as a result of tracking physiological indicators (urination amount, bladder internal pressure) in a civil bladder induction section, an accurate urination time point is indicated by a vertical dotted line.

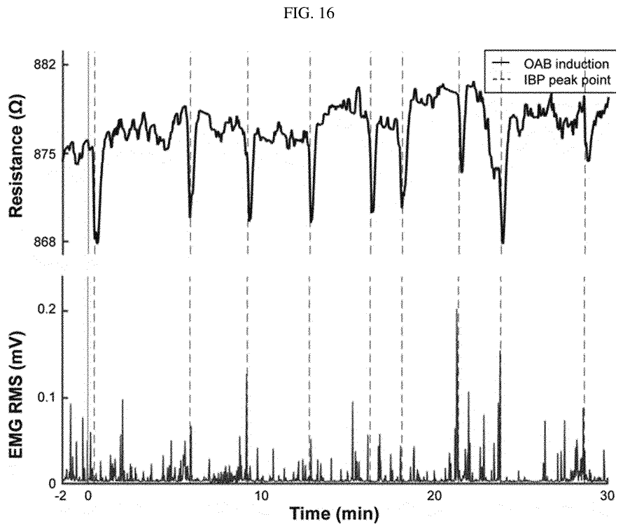

FIG. 16 shows measurement results of a tensile sensor and an EMG sensor in an overactive bladder induction region.

Referring to FIG. 16, there is an association between the urination time point and the measurement result.

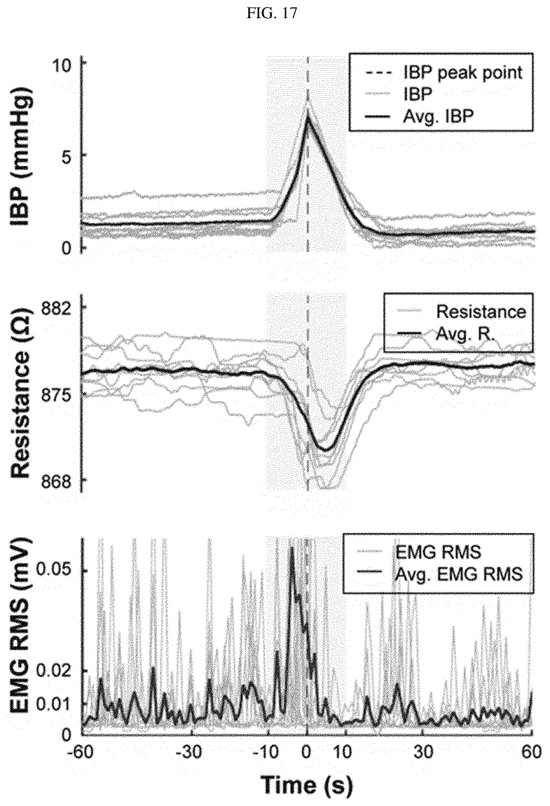

FIG. 17 is a result of extracting data near the urination time point and averaging it based on the urination time point.

Referring to FIG. 17, it can be seen that the sensor measurement results show a clear difference near the urination time point, and in particular, it can be seen that a higher EMG value is confirmed near the urination time point.

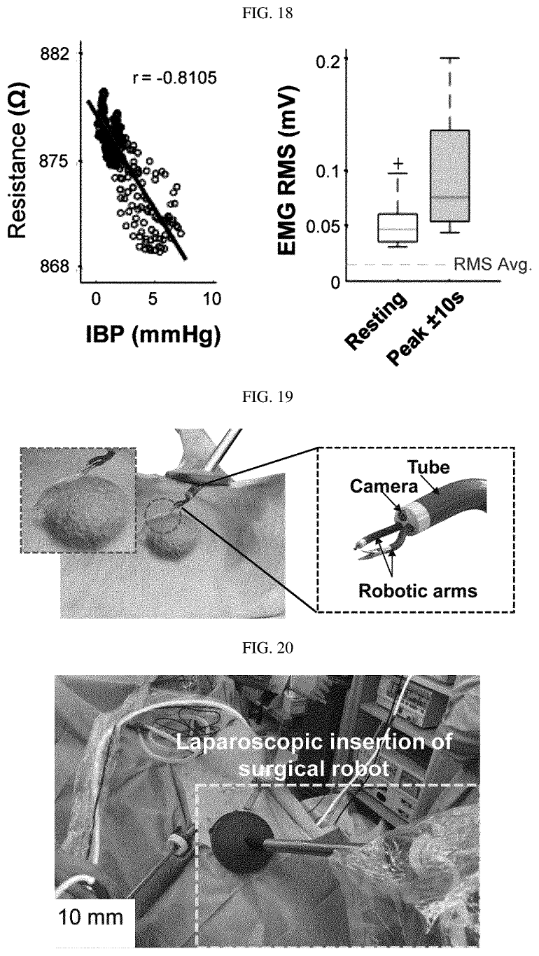

FIG. 18 is a result of quantitatively analyzing the result of FIG. 17.

Referring to FIG. 18, there is a significant correlation between the bladder internal pressure and the change in the tensile sensor near the urination time point (r=−0.8105), and the EMG activity is significantly higher near the urination time point compared to the general state. In particular, when the EMG signals are analyzed in the section of the general state and the section of the urination time point +−10 s, it can be seen that the average value is 1.81 times higher in the section of the urination time point, and thus, the EMG RMS signal point having a value of preferably 1.81, and most preferably 2×(average EMG RMS) or more can be determined as the urination time point.

By successfully analyzing the relationship between the EMG activation signal and the urination time point, it is possible to determine the urination time point through the EMG signal, and thus a bladder sensing system can be constructed.

FIG. 19 is an image of a surgical protocol in which a hydrogel-based implantable sensor developed by the present researchers is implanted using a minimally invasive laparoscopic robot implantable surgical instrument.

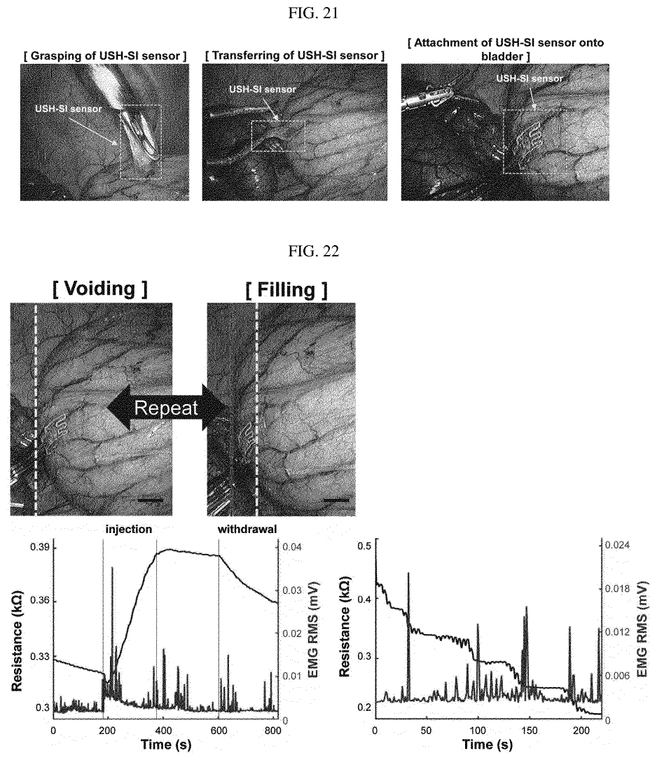

FIG. 20 is an image of an actual minimally invasive laparoscopic robot implant surgery using a pig, FIG. 21 is an image showing a surgical procedure in which a hydrogel-based implantable sensor developed by the present researchers using a laparoscopic robot implant surgery is implanted and attached to the bladder of a pig, and FIG. 22 is an image showing a repeated urination process of a pig, and the results of strain sensors and EMG sensors sensed in the urination process reflect the actual urination process of a pig.

Referring to FIGS. 19 to 22, the sensor according to the present invention can be effectively implanted into the bladder using a general minimally invasive laparoscopic robot to be bonded. In other words, a conventional silicon material having low adhesion has a hydrophobic surface, a large difference in Young's modulus (several MPa to several GPa) from a bio-organ (several Pa to several KPa), and no functional group capable of being attached to the surface of the bio-organ, and thus has poor adhesion properties. Due to these shortcomings, an additional fixation system is required to adhere to a bio-implantable sensor capable of analyzing physiological phenomena to the organ surface. Such a characteristic has a problem in that reliability of a sensor signal is deteriorated because an air layer is formed by being easily peeled off on a surface of a living body in order to analyze a physiological phenomenon in real time, and has a disadvantage in that a new fixture should be designed to be used for various organs.

However, due to high adhesive strength and biocompatible characteristics, the sensor attached by using the hydrogel which is a material having high biocompatibility according to the present invention may be attached to the detrusor muscle itself instead of the muscle around the bladder, thereby more directly progressing the measurement.

1. A real-time bladder sensing system using a bio-implantable sensor device, comprising:
a sensor unit attached to a bladder; and
an analysis unit configured to analyze a signal received from the sensor unit.

2. The real-time bladder sensing system of claim 1, wherein the sensor unit can simultaneously sense EMG and strain.

3. The real-time bladder sensing system of claim 1, wherein the sensor unit is formed on a hydrogel substrate containing polyacrylamide and is attached to the bladder through the hydrogel substrate.

4. The real-time bladder sensing system of claim 1, wherein the analysis unit filters a peak signal from the strain sensing signal and then predicts a urination point therefrom.

5. The real-time bladder sensing system of claim 1, wherein the analysis unit calculates a root-mean-square deviation of the EMG sensing signal to determine a urination time point and tracks a change in the number of urination time point thereby determining whether bladder is overactive or not.

6. The real-time bladder sensing system of claim 5, wherein the analysis unit determines an EMG RMS signal point having a value of 2×(average EMG RMS) or more as the urination time point.

7. The real-time bladder sensing system of claim 1, wherein the analysis unit receives EMG, and strain signals and integrates the received EMG and strain signals to predict a physiological phenomenon of the bladder.

8. The real-time bladder sensing system of claim 7, wherein the menstrual phenomenon of the bladder is a urination time point and whether bladder is overactive or not.

9. The real-time bladder sensing system of claim 3, wherein the EMG sensor comprises three electrodes formed on a radial island substrate, and the radial island substrate is bonded onto the hydrogel substrate.

10. The real-time bladder sensing system of claim 1, wherein a swelling prevention coating layer is formed on the sensor unit.

11. The real-time bladder sensing system of claim 9, wherein the swelling prevention coating layer comprises an acrylate-based material.

12. The real-time bladder sensing system of claim 10, comprising a modulus lower than that of the human bladder.

13. A sensor for a real-time bladder sensing system of claim 1.

14. The sensor for a real-time bladder sensing system of claim 13, wherein the sensor is attached to the bladder through a hydrogel-based adhesive.
